(19) United States
(12) Patent Application Publication
(10) Pub. No.: US 2007/0078181 A1
(43) Pub. Date: Apr. 5, 2007

(54) Use of a beta-3-agonist for the treatment of patients with spinal cord injury and suffering from renal and bladder complaints

(76) Inventor: Martin C. Michel, Amsterdam (NL)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/533,967

(22) Filed: Sep. 21, 2006

(30) Foreign Application Priority Data
Sep. 30, 2005 (EP) 05021420

(51) Int. Cl. A61K 31/24; A61K 31/195

(52) U.S. Cl. 514/534; 514/567

(57) ABSTRACT

This invention relates to the use of beta-3-adrenoceptor agonists for the prevention and treatment of kidney damage and/or functional bladder complaints, particularly bladder complaints of neurogenic origin as the result of spinal cord injury.

This invention relates to the use of beta-3-adrenoceptor agonists for the prevention and treatment of kidney damage and/or functional bladder complaints, particularly due to neurogenic causes as a result of spinal cord injury.

### PRIOR ART

The lower urinary tract consists of the bladder, the urethra, the associated muscles and the ligaments of the suspensory apparatus. The purpose of the bladder is to store the urine and evacuate it. The important factors for performing the storage function are not only the relaxation of the bladder muscle (detrusor muscle), but also the closure mechanisms provided by the neck of the bladder, the smooth muscle of the urethra and also the cross-striated muscle of the urethra and the pelvic floor. During the emptying of the bladder (micturition) the detrusor muscle contracts while the urethra and pelvic floor relax and the sphincter muscle of the bladder opens. These processes require complex control by the parasympathetic, sympathetic and somatic nervous system. Spinal cord injuries, e.g. transverse paralysis and other neurogenic causes, consequently lead to malfunctions in these areas. The detrusor often becomes unstable. One consequence of this may be that high pressures build up in the bladder, which are passed on to the kidneys. In some patients, urinary incontinence also develops. Even without incontinence these patients require treatment, as damage to the kidneys as a result of the high pressures is a serious threat. In particularly severe cases, incontinence may even be a deliberate choice, if the sphincter muscle has to be surgically cut in these patients, for example, so that the bladder can empty before high pressures build up.

Selective beta-3-adrenoceptor agonists have been discussed with a view to their suitability for various types of indications. These include, inter alia, obesity, diabetes and urinary incontinence. Since 1995 it has been known to use selective beta-3-adrenoceptor agonists in the treatment of urinary incontinence (EP 0 958 835). It has now been found that beta-3-adrenoceptor agonists can be used for the treatment or prophylaxis of kidney damage as a result of high pressures in the bladder or for the treatment of urinary incontinence in patients with spinal cord injury.

### PROBLEM OF THE INVENTION

The problem of the present invention is to treat damage to the kidneys as a result of high pressures in the bladder or to prevent such damage.

The invention also relates to the treatment of urinary incontinence in patients with spinal cord injury.

The invention also relates to the treatment of urinary incontinence of neurogenic origin as a result of Parkinson's disease, Alzheimer's disease, cerebro-cranial trauma, multiple sclerosis, inter alia, preferably as the result of Parkinson's disease, Alzheimer's disease or multiple sclerosis.

### DESCRIPTION OF THE INVENTION

According to the present invention a new pharmaceutical composition is provided which comprises at least one beta-3-adrenoceptor agonist in a pharmaceutically active amount as active ingredient.

a) Active Components

The preferred active components are mentioned hereinafter. To the extent that any pharmaceutically active compound is disclosed or claimed, it is expressly intended that all active metabolites which are produced in vivo are included, and it is expressly intended that all possible stereoisomers or tautomers are included. Also included are pharmacologically acceptable salts thereof. As examples of pharmaceutically active salts for the basic compounds the following are mentioned as acids for salt formation: acetic acid, benzenesulphonic acid (besylate), benzoic acid, p-bromophenylsulphonic acid, camphorsulphonic acid, carbonic acid, citric acid, ethanesulphonic acid, fumaric acid, gluconic acid, glutamic acid, hydrobromic acid, hydrochloric acid, hydriodic acid, isethionic acid, lactic acid, maleic acid, malic acid, mandelic acid, methanesulphonic acid (mesylate), mucinic acid, nitric acid, oxalic acid, pamoic acid, pantothenic acid, phosphoric acid, succinic acid, sulphuric acid, tartaric acid, p-toluenesulphonic acid and the like.

Insofar as it is necessary for completeness, the methods of synthesis of the compounds for which the prior art is mentioned and the dosages thereof are expressly included by reference to the prior art mentioned at the corresponding point.

The beta-3-adrenoceptor agonists used according to the invention are preferably phenoxyacetic acid derivatives. These are preferably selected from the following group according to formula I:

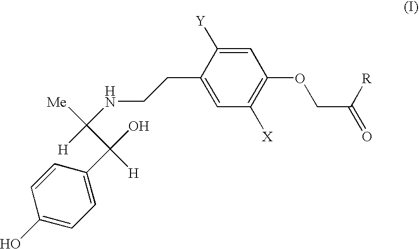

wherein

1) X═Br, Y═H, R═OH

2-[2-bromo-4-[2-[[(1 S,2R)-2-hydroxy-2-(4-hydroxyphenyl)-1-methylethyl]amino]ethyl]phenoxy]acetic acid,

2) X═Cl, Y═H, R═OH

2-[2-chloro-4-[2-[[(1S,2R)-2-hydroxy-2-(4-hydroxyphenyl)-1-methylethyl]amino]ethyl]-phenoxy]acetic acid,

3) X═Y═Cl, R═OH

2-[2,5-dichloro-4-[2-[[(1 S,2R)-2-hydroxy-2-(4-hydroxyphenyl)-1-methylethyl]amino]ethyl]phenoxy]acetic acid,

4) X═Y═H, R═OH

2-[4-[2-[[(1S,2R)-2-hydroxy-2-(4-hydroxyphenyl)-1-methylethyl]amino]ethyl]-2,5-dimethylphenoxy]acetic acid,

5) X═OH; Y═H; R═OH

2-[2-hydroxy-4-[2-[[(1S,2R)-2-hydroxy-2-(4-hydroxyphenyl)-1-methylethyl]amino]ethyl]phenoxy]acetic acid,

6) X═Cl; Y═H, R═OEt

ethyl-2-[2-chloro-4-[2-[[(1 S,2R)-2-hydroxy-2-(4-hydroxyphenyl)-1-methylethyl]amino]ethyl]phenoxy]acetate,

7) X═Cl; Y═Cl, R═OEt

ethyl-2-[2,5-dichloro-4-[2-[[(1S,2R)-2-hydroxy-2-(4-hydroxyphenyl)-1-methylethyl]amino]ethyl]phenoxy]acetate,

8) X=Me; Y=Me, R═OEt (−)-ethyl-2-[4-(2-{[(1S,2R)-2-hydroxy-2-(4-hydroxyphenyl)-1-methylethyl]amino}ethyl)-2,5-dimethylphenyloxy]acetate, and the corresponding hydrochloride.

9) X=Me; Y=Me, R═OH

(−)-2-[4-(2-{[(1S,2R)-2-hydroxy-2-(4-hydroxyphenyl)-1-methylethyl]amino}dimethylphenyloxy]acetic acid,

The compounds specified are disclosed in WO 00/02846 or WO 2003024916.

In addition, the following compounds are of interest: 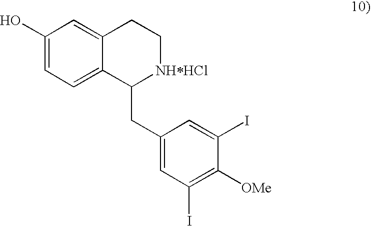
or the free base thereof.

Name: 1-(4-methoxy, 3.5-diiodophenyl-methyl-1.2.3.4-tetrahydro-isoquinolin-6-ol or the hydrochloride thereof, J. Med. Chem. 44 (2001) 1456.

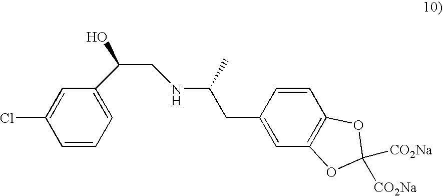

Name: Disodium-([R,R]-5-2-[[2-(3-chlorophenyl)-2-hydroxyethyl]-amino]propyl)-1, 3-benzodioxol-2,2-dicarboxylate or the hydrochloride thereof, J. Med. Chem. 44 (2001) 1456; Journal of Urology 165 (2001) 240.

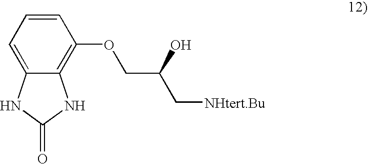

Name: 4-((3-N-tert.butylamino)-2-hydroxypropyloxy)-1,3-dihydro-benzoimidazol-2-one or the hydrochloride thereof, Journal of Urology 165 (2001) 240, J. Med. Chem. 44 (2001) 1456.

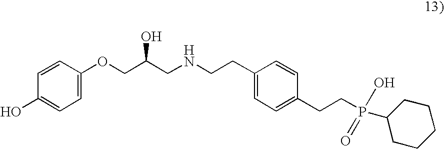

Name: cyclohexyl-[[4-[2-[[(2S)-2-hydroxy-3-(4-hydroxyl-phenoxy)-propyl]-amino]-ethyl]-phenoxy]-methyl]-phosphinic acid, J. Med. Chem. 44 (2001) 1456.

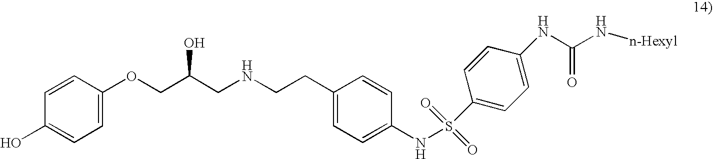

Name: (S)-4-[[(hexylamino)carbonyl]amino]-N-[4-[2-[[2-hydroxy-3-(4-hydroxy-phenoxy)-propyl]-amino]-ethyl]-phenyl]-benzenesulphonamide, J. Med. Chem. 44 (2001) 1456.

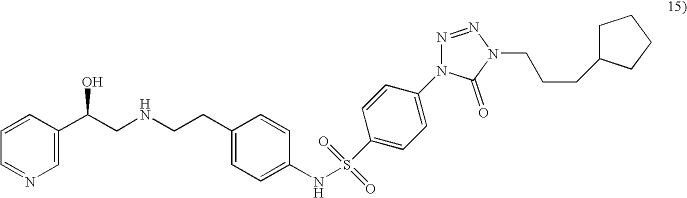

Name: (R)-4-[4-(3-cyclopentylpropyl)-4,5-dihydro-5-oxo-1H-tetrazol-1-yl]-N-[4-2-[[2-hydroxy-2-(3-pyridinyl)ethyl]amino]ethyl]-phenyl]-benzenesulphonamide or the hydrochloride thereof, J. Med. Chem. 44 (2001) 1456.

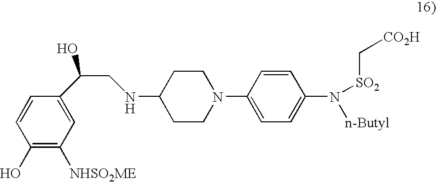

Name: 4-N-(N-(2-(4-hydroxy-3-methylsulphonamido-phenyl)-2-hydroxy-ethyl-4-amino)-piperidinyl-phenyl-(n-butylamino)-sulphonyl-acetic acid, J. Med. Chem. 44 (2001) 1456, Bioorg. Med. Chem. Lett. 9 (2001) 2045. 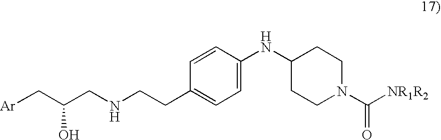
or the hydrochloride thereof
with
a) Ar=4-OHPh-O, R1=octyl, R2=H, name: 4-(4-(2-(3-(4-hydroxy-phenoxy)-2-hydroxy-propyl)-amino-ethyl)-anilino)-piperidinyl-carbonyl-N-octyl-amide;
b) Ar=4-OH, 3-methylsulphonylamidophenyl-O, R‘=2,5-difluorobenzyl, R2=H; Name: 4-(4-(2-(3-(4-hydroxy-3-methylsulphonylamidophenoxy)-2-hydroxy-propyl) -amino-ethyl)-anilino)-piperidinyl-carbonyl-N-(2,5-difluorobenzyl)-amide;
c) Ar=4-OH, 3-methylsulphonylamidophenyl, R1=2,5-difluorobenzyl, R2=H, Name: 4-(4-(2-(3-(4-hydroxy-3-methylsulphonylamidophenyl)-2-hydroxy-propyl) -amino-ethyl)-anilino)-piperidinyl-carbonyl-N-(2,5-difluorobenzyl)-amide;

(Bioorg. Med. Chem. Lett. 11 (2000) 3123). 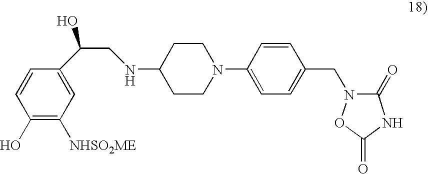
or the hydrochloride thereof

Name: 2-(4-N-(4-(2-(4-hydroxy-3-methylsulphonylamidophenyl)-2-hydroxy-ethyl)-amino) -piperidinyl)-benzyl)-[1,2,4]oxadiazolidine-3,5-dione, Bioorg. Med. Chem. Lett. 11(2001)981. 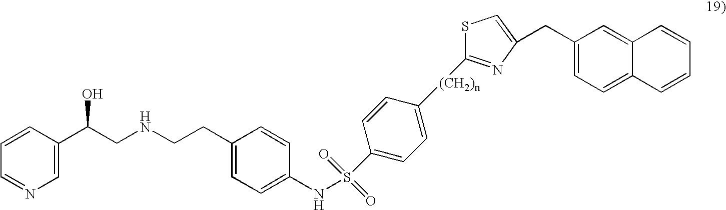
or the hydrochloride thereof

n maybe 0 or 1;

Name: (4-(4-(2-pyridinyl-2-hydroxyethylamino-ethyl)-anilino)-sulphonyl)-2-benzyl-4-naphth-2-ylmethyl-thiazole (n=1) and (4-(4-(2-pyridinyl-2-hydroxyethylamino-ethyl)-anilino)-sulphonyl)-2-phenyl-4-naphth-2-ylmethyl-thiazole (n=0), Bioor. Med. Chem. Lett. 10 (2000) 1971. 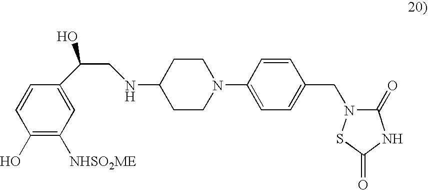
or the hydrochloride thereof.

Name: 2-(4-N-(4-(2-(4-hydroxy-3-methylsulphonylamidophenyl)-2-hydroxy-ethyl)-amino) -piperidinyl)-benzyl)-[1,2,4]thiadiazolidine-3,5-dione, Bioorg. Med. Chem. Lett. 11(2001)757. 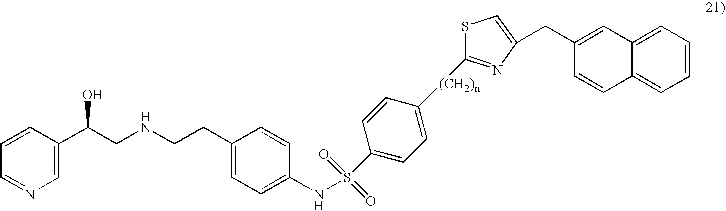
or the hydrochloride thereof.

n maybe 0 or 1.

Name: (4-(4-(2-pyridinyl-2-hydroxyethylamino-ethyl)-anilino)-sulphonyl)-2-benzyl-2-naphthyl-thiazole (n=1) and (4-(4-(2-pyridinyl-2-hydroxyethylamino-ethyl)-anilino)-sulphonyl)-2-phenyl-2-naphthyl-thiazole (n=0), Bioor. Med. Chem. Lett. 10 (2000) 1971. 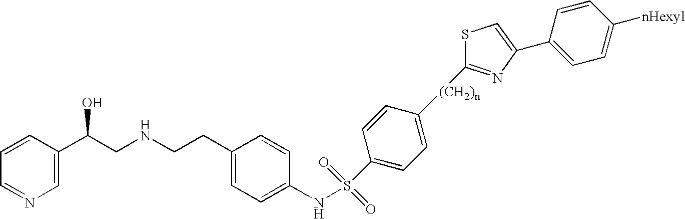
or the hydrochloride thereof.

Name: (4-(4-(2-pyridinyl-2-hydroxyethylamino-ethyl)-anilino)-sulphonyl)-2-benzyl-(4-n-hexyl -phenyl)-thiazole (n=1) and (4-(4-(2-pyridinyl-2-hydroxyethylamino-ethyl)-anilino)-sulphonyl)-2-phenyl-(4-n-hexyl-phenyl)-thiazole (n=0), Bioor. Med. Chem. Lett. 10 (2000) 1971. 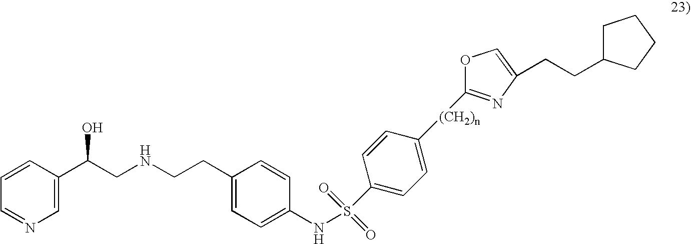
or the hydrochloride thereof.

Name: 2-(4-(4-(2-pyridinyl-2-hydroxyethylamino-ethyl)-anilino)-sulphonyl)-phenyl-4-(cyclopentyl -ethyl)-oxazole, Bioorg. Med. Chem. Lett. 10 (2000)1531. 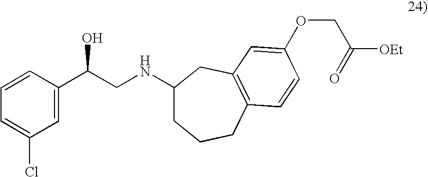
or the hydrochloride thereof.

Name: ethyl [R-(R*,S*)]-[[8-[[2-(3-chlorophenyl)-2-hydroxyethyl]amino]-6,7,8,9-tetrahydro-5H-benzocyclohepten-2-yl]oxy]-acetate, hydrochloride, 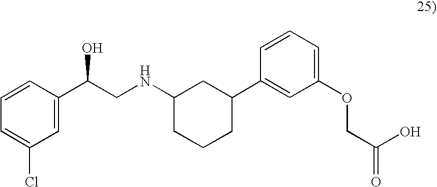
or the hydrochloride thereof.

Name: [1S-[1α,3β(S*)]]-3-[3-[[2-(3-chlorophenyl)-2-hydroxyethyl]amino]cyclohexyl]phenoxy]-acetic acid, monosodium salt, 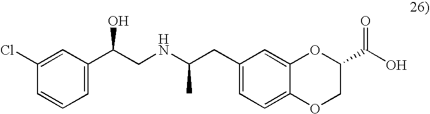
or the hydrochloride thereof.

Name: 6-[(2R)-2-[[(2R)-2-(3-chlorophenyl)-2-hydroxyethyl]-amino]propyl]-2,3-dihydro-(1.4-benzodioxine-2-carboxylic acid).

27)

2-(3-{[2-(3-chlorophenyl)-2R-hydroxyl-ethylamino]ethylamino}phenyl)thiophene-3-carboxylic acid or the hydrochloride thereof. 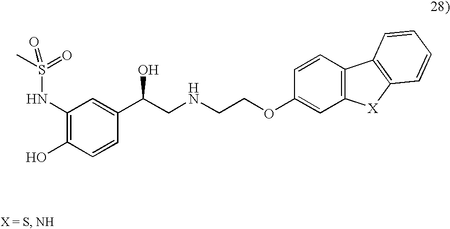
or the hydrochloride thereof.

Name: 3-(1-(4-hydroxy-3-methylsulphonylamido-phenyl)-1-hydroxy-ethyl-amino)-ethoxy)-dibenzothiophene and 2-(1-(4-hydroxy-3-methylsulphonylamido-phenyl)-1-hydroxy-ethyl-amino)-ethoxy)-9H-carbazole. 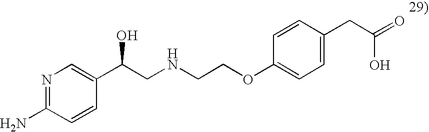
or the hydrochloride thereof.

Name: [4-[2-[[2-(6-aminopyridin-3-yl)-2(R)-hydroxyethyl]amino]ethoxy]phenyl]acetic acid. 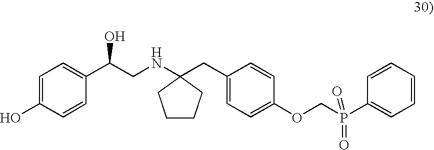
or the hydrochloride thereof.

Name: [[4-[[1-[[(2S)-2-hydroxy-3-(4-hydroxyphenoxy)-propyl]-amino]-cyclopentyl]- methyl]phenoxy]-methyl]-phenyl-phosphinic acid. 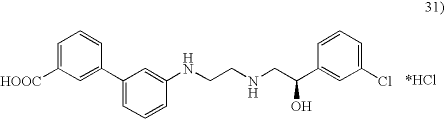
or the free base.

Name: [1,1′-biphenyl]-3-carboxylic acid, 3′-[[2-[[(2R)-2-(3-chlorophenyl)-2-hydroxyethyl]amino]ethyl]amino]-hydrochloride or solabegron. 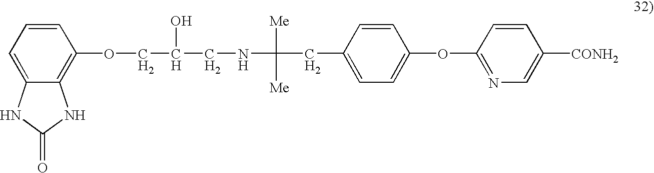
or the hydrochloride thereof

Name: 6-[4-[2-[[3-[(2,3-dihydro-2-oxo-1H-benzimidazol-4-yl)oxy]-2-hydroxypropyl]amino]-2-methyl-propyl]-phenoxy]-3-pyridinecarboxamide. 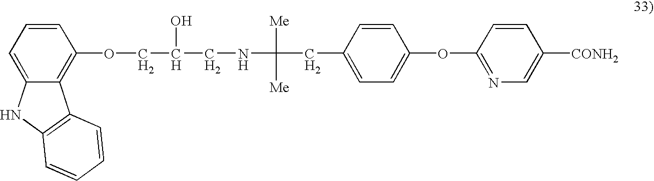
or the hydrochloride thereof.

Name: (S)-6-[4-[2-[[3-(9H-carbazol-4-yloxy)-2-hydroxypropyl]amino]-2-methylpropyl]phenoxy]-3-pyridinecarboxamide.

b) Dosage

In order to determine the optimum dose of the active substance, various basic conditions have to be taken into consideration such as for example the age and body weight of the patient, the nature and stage of the disease and the potency of the compound. This is deemed to be within the capabilities of the skilled man, and the existing literature on the components can be consulted in order to arrive at the optimum dose. The doses specified relate to the dosage after the adjustment phase.

The doses given hereinafter expressly include all the numerical values, both whole numbers and fractions, within the range specified. The data relate to adults. Paediatric doses may be lower.

More than one dose per day or two doses per day (e.g. 3, 4, 5 or 6 doses per day) are also expressly included herein.

In some cases a smaller amount than that specified may be sufficient while in other cases a larger total amount may be required.

The total daily dose may be taken in one go or in several portions depending on the treatment plan. The treatment plan may also prescribe intervals of longer than one day between the doses.

The average daily dose of the beta-3-agonist for an adult man is about 1 mg to 1000 mg, preferably 10 mg to about 750 mg per day, preferably 20 to 500 mg, more preferably 20 to 200 mg. This amount is preferably administered as a single dose per day or in two doses per day.

c) Formulations

The compositions of the present invention may conveniently be administered in a pharmaceutical composition which contains the active component in combination with a suitable carrier. Such pharmaceutical compositions may be prepared by methods and contain carriers which are well known in the art. Generally recognised textbooks are available to the skilled man for this purpose.

The compositions of the present invention may be administered parenterally (e.g. by intravenous, intraperitoneal, subcutaneous or intramuscular injection), topically, orally, intranasally, transdermally, rectally, by pulmonary or nasal inhalation, oral administration being particularly preferred. Of the oral formulations, those which are resistant to gastric juices are preferred. In such cases capsules or tablets resistant to gastric juices are preferred, and in both cases may be made with a coating which is resistant to gastric juices. The skilled man will find instructions for formulations resistant to gastric juices in the prior art.

Various formulating options are described below. The skilled man may choose a suitable formulation from them.

For oral therapeutic administration the composition according to the invention may be combined with one or more carriers and used in the form of tablets for swallowing, buccal tablets, sublingual tablets, sugar-coated tablets, sprays, powders, pastilles, coated tablets, granules, capsules, elixirs, suspensions, solutions, syrups, lozenges, chewing gums, foods and the like.

A spray may be prepared for example by grinding the particles of active substance to a suitable size.

Dilute sprays may be prepared by finely grinding the powdered substance with a non-toxic carrier material such as lactose and delivering it as a spray. Other suitable carrier materials for this purpose are other carbohydrates such as starch or mannitol. These sprays may optionally contain flavourings, preservatives, dispersing agents, colourings and other pharmacological adjuvants.

Capsules may be prepared from a powder of the kind described above or other powders, which are placed in a capsule, preferably a gelatine capsule, and the capsule is then sealed.

It is also possible for lubricants known from the prior art to be introduced into the capsule or used to seal the two parts of the capsule. The efficacy of a capsule when taken orally can be increased by the addition of disintegrating or solubilising substances such as, for example, carboxymethylcelluose, carboxymethylcellulose calcium, low-substituted hydroxypropylcellulose, calcium carbonate, sodium carbonate and other substances. The active substance may be present in the capsule not only as a solid but also in suspended form, for example in vegetable oil, polyethyleneglycol or glycerol using surface-active substances, etc.

Tablets may be prepared by compressing the powdered mixture and then processing it into granules, for example. The tablets may contain various excipients such as e.g. starches, lactose, sucrose, glucose, sodium chloride, urea for tablets for dissolving or injecting, amylose, various types of cellulose as described above and others. Glycerol or starch, for example, may be used as a moisture retaining agent.

The disintegrants used may be, for example, starch, alginic acid, calcium alginate, pectic acid, powdered agar-agar, formaldehyde gelatine, calcium carbonate, sodium bicarbonate, magnesium peroxide and amylose.

Anti-disintegrants or solution retardants which may be used include, for example, sucrose, stearin, solid paraffin (preferably with a melting point in the range from 50-52° C.), cocoa butter and hydrogenated fats.

Other disintegrants may be : corn starch, potato starch, alginic acid and the like.

Suitable absorption accelerators include, inter alia, quaternary ammonium compounds, sodium lauryl sulphate and saponins.

Ether may be used, for example, as a binder distributor and cetyl alcohol, glycerol monostearate, starch, corn starch, lactose, wetting agents (e.g. aerosol OT, Pluronics, Tweens), tragacanth gum, arabic gum, gelatine and others may be used as hydrophilising agents or disintegration accelerators.

Sucrose, fructose, lactose or aspartame may be used as sweeteners while peppermint, wintergreen oil, cherry flavouring etc may be used as flavouring agents. The above list is purely by way of example, and anyone skilled in the art could consider using other excipients known from the prior art.

Tablets may be produced by direct compression, for example.

Tablets and similar orally administered solid forms may be provided with coatins. For example, tablets, pills or capsules may be coated with gelatine, wax, shellac or sugar and the like. As already mentioned, formulations resistant to gastric juices are preferred for the oral preparations. Therefore, capsules or tablets resistant to gastric juices are preferred. In the case of a syrup or elixir, it may contain sucrose or fructose as sweetener, methyl- and propylparaben as preservative, a colouring and a flavouring agent such as cherry or orange flavouring.

It is also possible to prepare other formulations for oral administration such as solutions, syrups, elixirs etc. If desired the compound may be micro-encapsulated.

Parenteral administration may be achieved by dissolving the compound in a liquid and injecting it by subcutaneous, intramuscular or intravenous route. Suitable solvents include, for example, water or oily media.

In order to prepare suppositories the compound may be formulated with low-melting and water-soluble or water-insoluble materials such as polyethylene glycol, cocoa butter, higher esters (for example moerysthyl, palmitate) or mixtures thereof.

Naturally, any material used in the preparations of any dosage unit must be pharmaceutically acceptable and substantially non-toxic in the amounts used. In addition, the active components may be incorporated in preparations with delayed release and devices which, without being restricted thereto, include those based on osmotic pressures, in order to achieve the desired release profile. One-a-day formulations for each of the active components are particularly included.

Compositions and preparations of this kind should contain at least 0.001% of active compound. The percentage of the compositions and preparations may naturally vary and may appropriately make up between about 0.1 and about 100% of the weight of a given dosage unit. The quantity of active compound in therapeutically useful compositions of this kind is such that an effective dose is present.

d) Indications

Pharmaceutical composition according to the invention any of the compounds listed as beta-3-adrenoceptor agonists may be used for the treatment or prophylaxis of kidney damage as a result of pathogenic or involuntary build-up of high bladder pressures. In addition, each of these compounds may be used to treat urinary incontinence as a result of spinal cord injury. The spinal cord injury may be a consequence of both accidents and also of diseases, e.g. tumours. According to the invention urinary incontinence as the result of Parkinson's disease, Alzheimer's disease, cerebro-cranial trauma, multiple sclerosis may also be treated. Complaints resulting from Parkinson's disease, Alzheimer's disease or multiple sclerosis are preferred.

Thus, a further embodiment of the present invention comprises using the composition according to the invention to prepare a drug for treating or preventing any of the indications mentioned in the preceding paragraph.

The above diseases or disorders are treated by administering a therapeutically effective amount of the composition according to the invention to a mammal. In most cases this is a human being but the treatment of farm animals (e.g. cattle) and domestic animals (e.g. dogs, cats and horses) is also expressly covered. For use in veterinary medicine the dosages used may be different from those specified herein.

It is expected that the new composition will provide rapid relief for those suffering from the above diseases and disorders with a minimum amount of harmful side effects.

e) Combinations

For use according to the invention the beta-3-adrenoceptor-agonist may also be combined with other active substances.

The following are mentioned as preferred examples of combination partners for the treatment of urinary incontinence:

alpha blockers such as tamsulosin, tamsulosin hydrochloride, alfuzosin, bunazosin, doxazosin, indoramin, naftopidil, prazosin, terazosin, urapidil, silodosin, moxisylyt, metazosine, fiduxosin, upidosine, SNAP-5089 (methyl (5-(N-(3-(4,4-diphenylpiperidin-1-yl)propyl)carbamoyl) -2,6-dimethyl-4(R)-(4-nitrophenyl)-1,4-dihydropyridine-3-carboxylate), AIO-8507L,SL-890591 ((2-(3-(4-(5-chloro-2-methoxyphenyl)piperazin-1-yl)propylamino)pyrimidine-4-carboxamide fumarate), RS-100329 (5-methyl-3-(3-(4-(2-(2,2,2-trifluoroethoxy)phenyl)piperazine-1-yl)propyl) pyrimidin-2,4(1H,3H)-dione hydrochloride);

antimuscarinics such as (S)-N-{3-[4-(2-(2,3-dihydrobenzofuran-5-yl)-1-methylethyl)-ethylamino]-methyl-piperidin-1-yl]-3-oxopropyl}-methanesulphonamide, 1-azabicyclo[2,2,2]oct-4-yl [1,1′-biphenyl]-2-ylcarbamate monohydrochloride, 2-methyl-alpha,alpha-diphenyl-1H-imidazole, AH-9700, N-(4-methylamino-benzyl)-piperidin-4-yl benzhydryl-carbamate, bethancholchloride, darifenacin, darifenacin chloride, dicyclomin hydrochloride, emepronium chloride, fesoterodine, FK-584, hyoscyamine sulphate, imipramine hydrochloride, oxybutynin chloride, S-oxybutynin chloride, ipratropium, J-104135, N-[2-(2,3-dihydrobenzofuran-5-yl)-1-methylethyl]-N-ethyl-(1-methanesulphonylpiperidin-4-ylmethyl)-amine, N-ethyl-N-[2-(4-methoxyphenyl)-1-methylethyl)-[1-(dimethylaminocarbonyl)-piperidin-4-ylmethyl]-amine, oxybutynin, propanthelin bromide, propiverine, propiverine chloride, revatropate chloride, solifenacin, temiverin, temiverine chloride, terodiline chloride, tolteridine tartrate, tolterodine, trospium, trospium chloride, vamicamide chloride.

For the therapeutic or prophylactic treatment of kidney damage suitable combination partners are ACE-inhibitors, angiotensin receptor blockers, renin inhibitors, calcium antagonists, diuretics or beta 1- and/or beta 2 adrenoceptor antagonists, endothelin-receptor-antagonists. Preferred combination partners are ACE-inhibitors, angiotensin receptor blockers/angiotensin II antagonists and diuretics.

Examples of ACE inhibitors include: alacepril, alatriopril, altiopril calcium, ancovenin, benazepril, benazepril hydrochloride, benazeprilat, benzoylcaptopril, captopril, captopril-cystein, captoprilglutathione, ceranapril, ceranopril, ceronapril, cilazapril, cilazaprilat, delapril, delaprildiacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, llisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, guinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril, zofenoprilat.

Examples of angiotensin II antagonists include: candesartan, candesartan cilexetil, losartan, valsartan, irbesartan, tasosartan, telmisartan, eprosartan.

An example of a diuretic is: hydrochlorothiazide.

What is claimed is:

1. A method for treating or reducing the incidence of kidney damage or kidney disease resulting from pathogenically high pressure or high pressure in the bladder caused by malfunction of the detrusor, which method comprises the administration of a therapeutically or prophylactically effective amount of a beta-3-adrenoceptor agonist.

2. A method for treating a bladder disease or condition resulting from spinal cord injury, which method comprises the administration of a therapeutically or prophylactically effective amount of a beta-3-adrenoceptor agonist.

3. A method for treating a bladder disease or condition resulting from Parkinson's disease, Alzheimer's disease, cerebro-cranial trauma or multiple sclerosis, which method comprises the administration of a therapeutically or prophylactically effective amount of a beta-3-adrenoceptor agonist.

4. A method for treating a bladder disease of neurogenic origin, which method comprises the administration of a therapeutically or prophylactically effective amount of a beta-3-adrenoceptor agonist.

5. The method according to claim 1, 2, 3 or 4, wherein the beta-3-adrenoceptor-agonist is a compound of the formula I 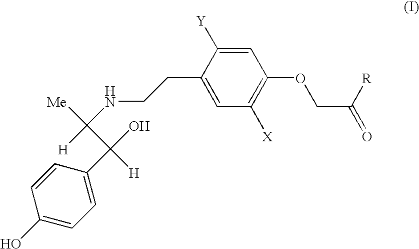
wherein X═H, Cl, Br, OH, methyl, Y═H, Cl, Br, OH, methyl, R═OH, methoxy, ethoxy or a pharmaceutically acceptable salt thereof.

6. The method according to claim 5, wherein:
X═Br, Y═H, R═OH.

7. The method according to claim 5, wherein:
X═Cl, Y═H, R═OH.

8. The method according to claim 5, wherein:
X═Y═Cl, R═OH.

9. The method according to claim 5, wherein:
X═Y═H, R═OH.

10. The method according to claim 5, wherein:
X═OH; Y═H; R═OH.

11. The method according to claim 5, wherein:
X═Cl; Y═H, R═OEt or the corresponding hydrochloride.

12. The method according to claim 5, wherein:
X═Cl; Y═Cl, R═OEt or the corresponding hydrochloride.

13. The method according to claim 5, wherein:
X=Me; Y=Me, R═OEt or the corresponding hydrochloride.

14. The method according to claim 5, wherein:
X═Me; Y═Me, R═OH.

15. The method according to claim 5, wherein the beta-3-adrenoceptor-agonist is used in an amount from 10 mg to 750 mg.
